(19) United States
(12) Patent Application Publication
(10) Pub. No.: US 2003/0118657 A1
(43) Pub. Date: Jun. 26, 2003

(54) Treatment of disease states characterized by excessive or inappropriate angiogenesis

(76) Inventors: Jennifer L. West, Pearland, TX (US); J. Donald Payne, Spring, TX (US)

Correspondence Address: ERIC P. MIRABEL, 3783 DARCUS, HOUSTON, TX, 77005, US

(21) Appl. No.: 10/308,512

(22) Filed: Dec. 3, 2002

Related U.S. Application Data
(60) Provisional application No. 60/336,824, filed on Dec. 4, 2001.

(51) Int. Cl. A61K 39/395; A61K 38/18; A61K 9/14

(52) U.S. Cl. 424/489; 514/12; 424/178.1

(57) ABSTRACT

Disclosed is a method for reducing excessive or inappropriate neovasculature, including nevasculature in the eye which interferes with or has potential to interfere with vision, for example, that associated with diabetic retinopathy or macular degeneration. The regions of the neovasculature are targeted with nanoparticles, including metal nanoshells, which are then irradiated, preferably with a laser, to heat them and ablate the undesired blood vessels. The nanoparticles are targeted to the neovasculature by linking them with a targeting agent, including, for example, antibodies, antibody fragments, receptor binding proteins or other proteins or molecules including growth factors.

This Application claims the benefit of U.S. Provisional Application No. 60/336,824, filed on Dec. 3, 2001.

### BACKGROUND OF THE INVENTION

Certain disease states and conditions, including macular degeneration, diabetic retinopathy, cancer and healing wounds, are characterized by excessive or inappropriate angiogenesis. Macular degeneration and diabetic retinopathy can both lead to blindness or deterioration of vision. In both these conditions, new blood vessels which proliferate in the retina are the main cause of vision impairment. In cancer, the tumor promotes the growth of new blood vessels to support the growth of the tumor. Angiogenesis arising in connection with wounds may impair healing.

Macular degeneration relates to a breakdown of the macula, the light-sensitive part of the retina responsible for the sharp, direct vision needed for activities including reading or driving. Macular degeneration is more common in people over age 65, and whites and females are at highest risk. Most cases of macular degeneration are related to aging (age-related macular degeneration), but it also can occur as a side effect of some drugs, and it appears to run in families.

Age-related macular degeneration (“AMD”) is diagnosed as either dry (atrophic) or wet (exudative). The dry form is more common than the wet, with about 90% of AMD patients diagnosed with dry AMD. The wet form of the disease usually leads to more serious vision loss. Wet AMD affects approximately 10% of people with AMD, but accounts for approximately 90% of all severe vision loss from AMD (National Eye Institute).

With wet AMD, new poorly formed blood vessels grow beneath the retina (from the choroids) and leak blood and fluid into the retina and subretinal space. This leakage causes retinal cells to die, promotes scarring of the fovea (central macula), and the scarring creates blind spots in central vision.

Diabetic retinopathy is a complication of diabetes. In proliferative diabetic retinopathy, new blood vessels grow on the surface of the retina. These new blood vessels can lead to serious vision problems because they can break and bleed into the vitreous humor. Proliferative retinopathy is a serious form of the disease and can lead to blindness. Diabetic retinopathy is the leading cause of blindness in adults 20 to 74 years of age.

In diabetic patients, hyperglycemia (high blood glucose levels) can result in oxygen starvation (“ischemia”) of the retina. Retinal ischemia is believed to stimulate the release of angiogenic factors that induce proliferation of additional blood vessels (“neovascularization”). These blood vessels are fragile and may break and bleed into the surrounding retinal tissue. This also leads to scarring within the eye, which may pull the retina forward, causing it to detach and vision to be completely lost.

As oxygen deprivation begins, excess growth factors are released to promote neovascularization. Among these various growth factors, vascular endothelial growth factor (“VEGF”) is released and migrates to the endothelial cells lining these blood vessels. Retinal blood vessels have three times as many receptors for VEGF as vessels elsewhere, and the oxygen deficit dramatically raises VEGF levels. VEGF is believed to play a major role in triggering neovascularization.

The elimination or reduction of such newly generated blood vessels offers a way to treat or ameliorate macular degeneration or diabetic retinopathy. Current methods of treatment of wet macular degeneration and proliferative diabetic retinopathy involve destruction of the neovasculature. In one currently used method, termed laser photocoagulation therapy, a surgeon uses a laser to coagulate tissue, sealing and destroying leaking blood vessels. Laser photocoagulation involves brief exposures to tiny spots of intense laser light to the area occupied by abnormal blood vessels. The light energy is absorbed by pigment in retinal pigment epithelium (“RPE”) cells and converted to heat energy that cauterizes and destroys the abnormal blood vessels. This process often leads to regression of new blood vessel formation.

However, laser photocoagulation destroys cells surrounding the proliferating capillaries, resulting in visual impairment at the treatment site. As a result, this therapy may be repeated only a limited number of times before seriously degrading visual acuity. Typically, only patients with certain well-defined vessel growth (termed “classical”) in particular areas are recommended for laser photocoagulation treatment. In other patients, where the abnormal blood vessels are not localized or are located beneath the center of the macula, the treatment would tend to destroy the overlying central retina and result in loss of central vision.

Photodynamic therapy is another method used to treat these disorders, and involves injection of a photosensitive drug, such as verteporfin, followed by irradiation of the macula or retina with low intensity laser light to activate the drug. The drug becomes concentrated in the choroidal neovasculature (CNV). It is postulated that the drug absorbs the laser light and releases reactive oxygen intermediates that selectively damage the abnormal blood vessels, while doing less damage to the overlying retina. Results of an experimental study were published in the October 1999 issue of Archives of Opthalmology, and the FDA approved of verteporfin for treating wet AMD, under the trade name VISUDYNE® in April 2000. It is only indicated for patients whose new blood vessels are characterized as “predominantly classic” (a well-defined area of neovascularization): a group comprising about 40% to 60% of the new wet AMD patients. In this treatment procedure, VISUDYNE® is injected systemically, then activated in the CNV by shining a laser into the eye. In clinical trials, 67% of patients found that either their vision loss stabilized or that their vision improved.

However, a disadvantage of this treatment is that photodynamic dyes are not specific to the neovasculature in the eye, but dissipate throughout the body tissues. It is recommended that patients not be in sunlight for five days after VISUDYNE® treatment. Additionally, Photodynamic therapy agents as a class have been reported to result in DNA damage, including chromosomal aberrations and mutations.

Researchers are also investigating the use of indocyanine green, an infrared dye, and near infrared light for treatment of macular degeneration and diabetic retinopathy. See, for example, Costa, et al., Am Journal of Ophthalmology, October 2001; 132(4):557-65. This is still an experimental and unproven treatment, and also is likely to have many of the same disadvantages as VISUDYNE® treatment.

Vascular endothelial growth factor (VEGF) is secreted not only from ischemic tissue, but also from many types of cancerous cells. VEGF regulates angiogenesis by binding to specific receptors on nearby blood vessels, causing new blood vessels to form via endothelial cell proliferation and migration. Others have investigated the use of anti-VEGF receptor antibodies (See U.S. Pat. No. 6,342,219) or antisense therapy (See U.S. Pat. No. 6,410,322; International Patent Application No. WO 0231141) as a treatment of excessive angiogenesis, including macular degeneration, diabetic retinopathy, inappropriate wound healing and/or cancer. Such anti-angiogenesis factors suffer a disadvantage in that arresting growth systemically may have undesirable effects on healthy tissue elsewhere.

### SUMMARY OF THE INVENTION

The invention relates to reducing or eliminating excessive or inappropriate neovasculature through the use of nanoparticles to deliver heat sufficient to disrupt or ablate such neovasculature, where the nanoparticles include nanoshells as disclosed in U.S. Pat. No. 6,344,272 (incorporated by reference), metal colloids as disclosed in U.S. Pat. No. 5,620,584 272 (incorporated by reference), fullerenes and derivatized fullerenes, as disclosed in U.S. Pat. Nos. 5,739,376; 6,162,926; 5,994,410 [See also, Diederich, F. et al., Science, Vol. 252, pages 548-551 (1991) and Smart, C. et al., Chem. Phys. Lett., Vol. 188, No. 3, 4, pages 171-176 (Jan. 10, 1992) disclosing fullerenes other than C₆₀] all of which are incorporated by reference, as well as nanotubes including single walled nanotubes, as disclosed in U.S. Pat. No. 6,183,714 (incorporated by reference), which can also be derivatized. A nanoshell may include a core substrate material having a smaller dielectric permittivity than the preferred metallic material of the outer shell. The nanoparticle may be conjugated or associated with a targeting molecule, where the targeting molecule targets the nanoparticle to regions of neovasculature associated with a disease state. Such targeting molecules can be antibodies, antibody fragments, receptor binding proteins or other proteins or molecules including growth factors. The nanoparticles may also be conjugated with a polymer to reduce opsonization of the nanoparticles. Suitable polymers include polyethylene glycol The targeting molecules may be conjugated to the nanoparticles by conjugation to the distal end of the polymer.

The treatment methods of the invention are well-suited for treating diseases involving undesired neovasculature, including that associated with cancer (as disclosed in U.S. Patent Application Publication No. 20020103517, incorporated by reference), with inappropriate wound healing, or neovasculature associated with the eye which is, or has potential to be, vision impairing. Following localization of the nanoparticles at the areas of neovasculature, the region is irradiated with a laser, at a wavelength minimally absorbed by the surrounding tissue but preferentially absorbed by the nanoparticle so as to cause the generation of heat by the nanoparticles sufficient to cause disruption of the neovasculature but with minimal disruption or ablation of the surrounding tissue. Such wavelength is preferably between 700 nm and 1300 nm and more preferably between 750 nm and 1100 nm. Such preferential absorption results in the nanoparticles absorbing the radiation and converting it to heat with a higher efficiency than radiation is absorbed by the surrounding tissue. This can cause a two-to-one or greater rise in temperature of the nanoparticles than in the surrounding irradiated tissue.

Thus, there is a provided a non-invasive treatment for angiogenesis associated with a disease state, including angiogenesis-related vision impairing conditions, including those associated with macular degeneration and diabetic retinopathy. Because the degree of heat is controlled and it is localized to the target area, there is expected to be little damage to the surrounding tissues and/or the retina, in contrast to the conventional laser photocoagulation therapy.

The making and using of the invention are described further below, with reference to the drawings.

### BRIEF DESCRIPTION OF THE DRAWINGS

FIG. 1 is a partially cut-away view of a nanoshell suitable for use with the invention.

FIG. 2 is a sectional depiction of the nanoshell of FIG. 1.

FIG. 3 is a sectional depiction of another type of nanoshell suitable for use in the invention.

FIG. 4 is a sectional depiction of yet another type of nanoshell suitable for use in the invention.

FIG. 5 is graphical depiction of a plasmon resonance peak, showing a plot of intensity against wavelength.

### DETAILED DESCRIPTION OF THE INVENTION

A. Embodiments of Nanoshells

The nanoparticles suited for use in the invention include metal nanoshells. The nanoparticles can be conjugated to or bound with any targeting molecule, including antibodies, antibody fragments, receptor binding peptides, growth factors and other proteins. As noted above, one example of a nanoshell suitable for use in the invention has a core substrate material with a smaller dielectric permittivity than the metallic material of the outer shell. Other embodiments of metal nanoshells are described further below and in U.S. Pat. No. 6,344,272, hereby incorporated by reference.

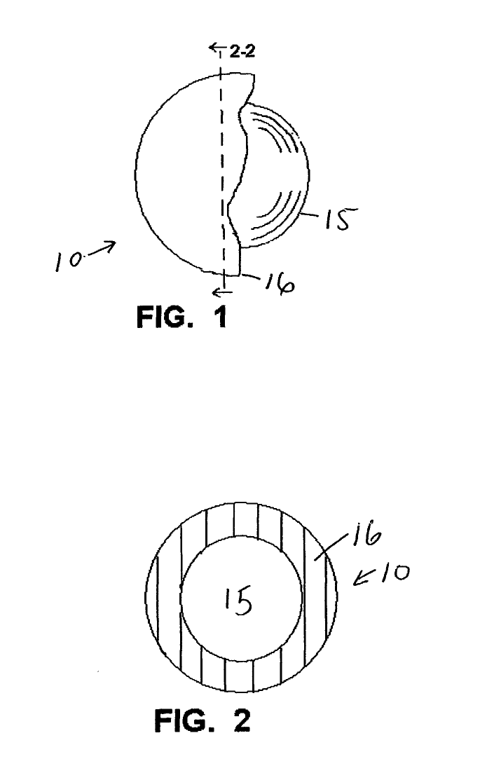

Referring initially to FIGS. 1 and 2, according to a preferred embodiment of the present invention, a nanoshell 10 includes a core 15 and a shell 16. Nanoshell 10 is preferably a nanoparticle having a size between about 1 nanometer and about 5 microns. Nanoshell 10 is preferably spherical in shape, but may have any geometrical shape, such as cubical, cylindrical, hemispherical, elliptical, and the like. The size of nanoshell 10 is preferably defined by the average diameter of nanoshell 10.

The average diameter of an object, such as nanoshell 10, having a surface defining the extent of the object, is the angular average of the distance between opposing regions of the surface through a fixed point located interior to the object. For an object described by a radial coordinate system centered at the fixed point, the average is over both the radial angle θ and the aziumuthal angle φ. That is, the average diameter <D> of the diameter D(θ, φ) is given by <D>=(θdφD(θ, φ))/4π².

Core 15 is also preferably spherical, but may have any geometrical shape, such as cubical, cylindrical, hemispherical, elliptical, and the like. The average diameter of core 15 is preferably between about 1 nanometer and about 5 microns, more preferably between about 10 nanometers and about 2 microns.

Core 15 preferably includes a substrate material, i.e., any material that has a smaller dielectric permittivity than preferred materials for outer shell 16. Preferably, the substrate material either is or includes a dielectric material, for example, a semiconducting material. Suitable substrate materials include, but are not limited to, silicon dioxide (also termed silica), titanium dioxide, polymethyl methacrylate, polystyrene, gold sulfide, cadmium sulfide, gallium arsenide and dendrimers. In some embodiments, the substrate material is arranged as a surface layer of core 15.

Shell 16 is preferably layered on core 15, and may be arranged such that the inner surface of shell 16 contacts the outer surface of core 15. Alternatively, the contact between core 15 and shell 16 may occur only between portions of core 15 and shell 16.

The inner and outer surfaces of shell 16 can each be spheroidal, or one or both surfaces can have an alternative shape, including cubical, cylindrical, hemispherical or elliptical. Shell 16 preferably includes a metallic material, which may be a single element or an alloy, more preferably a binary alloy. As used herein, metals include those elements disclosed in the USPTO Manual of Classification as metals. Both the old IUPAC notation, with Roman numerals, and the new notation, with Arabic numbers will be used herein. See, for example Lewis, Richard J., Sr., “Hawley's Condensed Chemical Dictionary” (1997, John Wiley and Sons), the inside front cover page, hereby incorporated herein by reference, for a comparison of notations. In particular, Group I metals include Group 1 metals (Li, Na, K, Rb, Ca, and Fr) and Group 11 metals (Cu, Ag, and Au). Group II metals include Group 2 metals (Be, MG, Ca, Sr, Ba, and Ra) and Group 12 metals (Zn, Cd, and Hg). Group III metals include Group 3 metals (Sc and Y) and Group 13 metals (Al, Ga, In, and Tl). Group IV metals include Group 4 metals (Ti, Zr, and Hf) and Group 14 metals (Ge, Sn, and Pb). Group V metals include Group 5 metals (V, Nb, and Ta) and Group 15 metals (As, Sb, and Bi). Group VI metals include Group 6 metals (Cr, Mo, and W) and Group 16 metals (Po). Group VII metals include Group 7 metals (Mn, To, and Re). Group VIII metals include Group 8 metals (Re, Ru, and Os), Group 9 metals (Co, Rh, and Ir), and Group 10 metals (Ni, Pd, and Pt). A metallic material forming shell 16 preferably is selected from the elements of Groups I and VIII. More preferably, the metallic material is selected from among copper (Cu), silver (Ag), gold (Au), nickel (Ni), platinum (Pt), palladium (Pd), and iron (Fe). Alternatively, in some embodiments, the metallic material includes a synthetic metal. A synthetic metal is defined herein as an organic or organometallic material that has at least one characteristic property in common with a metal including, for example, electrical conductivity. Thus, synthetic metals include conducting polymers, such as polyacetylene and polyanaline. Shell 16 may, therefore, include one or more of an elemental metal, an alloy and a synthetic metal.

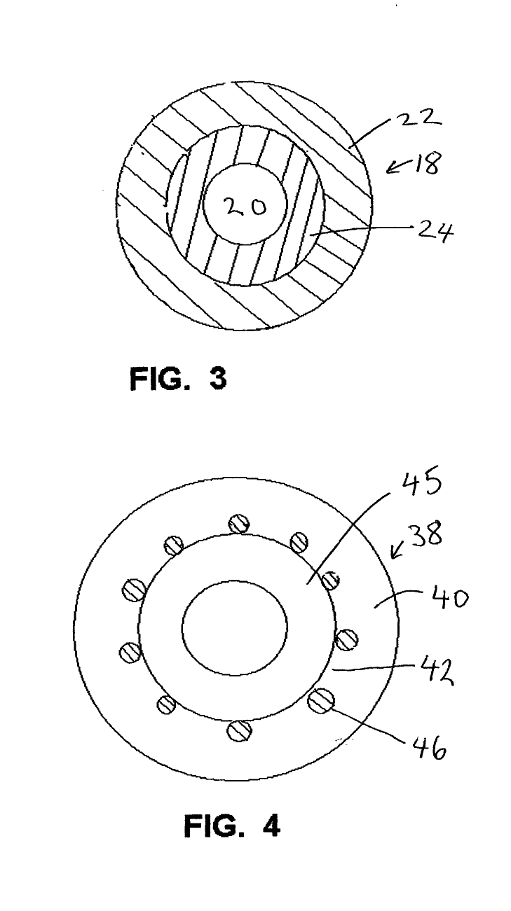

Referring now to FIG. 3, this embodiment shows an intermediate material layer 24 disposed between shell 22 and core 20 of a nanoshell 18. Layer 24 preferably includes a functionalizing material that is adapted to bind core 20 to a shell 22. Thus, the presence of the intermediate layer 24 functionalizes the core, allowing a metallic material to be coated directly onto the surface of functionalized core 26, which is formed by core 20 and layer 24.

Preferably, the functionalizing material of layer 24 is a metallic material adapted to receive the primary metallic material forming shell 22, for example by reduction of primary metallic material onto the functionalizing material. The functionalizing material is preferably tin. Alternatively, titanium, which has similar reduction properties to tin, could be used. A portion of the functionalizing material forming layer 24 is the reaction product of ions of the functionalizing material with hydroxyl groups at the surface of a silica core, and may also be the reaction product of reduction from solution of ions of the functionalizing material onto the functionalizing material bound to the core.

Intermediate layer 24 may also include a plurality of linker molecules arranged such that one end of each linker molecule binds to core 20 and the other end of each linker molecule binds to shell 22. One end of a linker molecule includes a first functional group which binds to material contained in core 20 and the other end of the linker molecule includes a second functional group which binds to a material contained in shell 22. Aminopropylsilanetriol, which is the hydrolyzed form of aminopropyltriethoxysilane (APTES), is among the linker molecules suited to linking a metallic shell to a silica core. Others include the hydrolyzed form of any suitable amino silane, including aminopropyltrimethoxy silane, diaminopropyl-diethoxy silane and 4-aminobutyldimethylmethoxy silane, or the hydrolyzed form of any suitable thio silane, including mercaptopropyltrimethoxy silane.

The silanol groups at one end of aminopropylsilanetriol have an affinity for silica, in particular hydroxyl groups at the surface of silica. Thus, a silanol linkage between core 20 and aminopropylsilanetriol is obtained from the reaction of a silanol group of aminopropylsilanetriol with a hydroxyl group on core 20, with elimination of water. An amino group at the other end of aminopropylsilanetriol has an affinity for metallic materials. Thus, an amino linkage between shell 22 and aminopropylsilanetriol is obtained from the reaction of aminopropylsilanetriol with shell 22.

Referring now to FIG. 4, a composite particle 38 includes a shell 40 that includes a precursor metallic material 42 that may be different from a metallic material 44 that principally forms shell 40. Precursor material 42 provides nucleation sites for the formation of shell 40. Precursor material 42 preferably includes colloidal particles 46 distributed over the surface of core 48. Colloidal particles 46 may be embedded into shell 40, but are preferably bound to intermediate layer 45. Colloidal particles 46 may be bound to linker molecules in intermediate layer 45. For example, gold colloidal particles 46 may bind to aminopropylsilanetriol and serve as nucleation sites for a silver shell 40. Alternatively, tin colloidal particles may extend from an intermediate layer 45 that includes tin. In an exemplary arrangement, as disclosed in U.S. Patent Application Publication Number 20020061363, filed Sep. 27, 2001, which is incorporated herein by reference, subparticles were made including gold colloidal precursor particles having a size between about 1 and about 3 nanometers that served as nucleation site for a silver shell having a thickness between about 10 nanometers and about 20 nanometers. It was been observed that, for this arrangement, the plasmon resonance associated with the silver shell was consistent with a pure silver shell, and the presence of the gold colloids was not significant.

Referring again to FIGS. 1 and 2, nanoshell 10 has a plasmon resonance associated with shell 16. The plasmon resonance is determined by detecting a peak in an absorption or a scattering spectrum. The peak is preferably determined by plotting intensity as a function of wavelength. Further, the plot may be a plot of intensity as a function of any other spectroscopic variable, such as wavenumber (e.g. cm⁻¹) or frequency (e.g. mHz and the like). A wavelength λ, wavenumber n, and frequency v are conventionally related as λ=vᵣ/v=1/n, where vᵣ is the velocity of propagation of the radiation. For propagation in a vacuum, vᵣ=c, the speed of light. When the spectrum is an absorption spectrum the intensity is the intensity of radiation that is absorbed. When the spectrum is a scattering spectrum, the intensity is the intensity of radiation that is scattered.

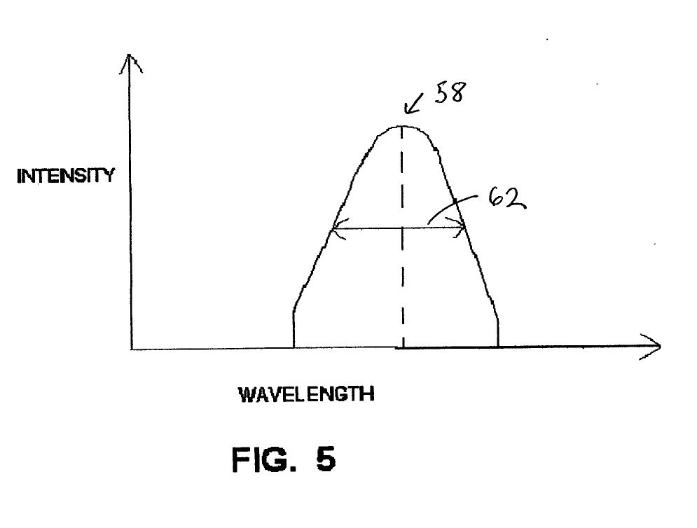

Referring now to FIG. 5, a plasmon resonance peak 58 preferably has a peak wavelength 60 and a peak width 62. Peak wavelength 60 is the wavelength at which plasmon resonance peak 58 is at a maximum. Peak width 62 is the full width half maximum of plasmon resonance peak 58. Peak width 62 may include contributions from both homogenous and non-homogeneous line broadening. Homogeneous line broadening occurs in part as a result of electron collisions. Peak width 62 therefore depends in part on the shell electron mean free path.

For nanoshells for use in the invention, peak wavelength 60 preferably is red-shifted, (that is a shift to longer wavelength) from the peak wavelength of a colloidal particle made of the same material as the primary material forming shell 16. Gold and silver are exemplary metallic materials for use in shell 16. When shell 16 includes principally silver, nanoshell 10 may have a plasmon resonance with a peak wavelength from about 400 nanometers to about 20 microns. In contrast, the peak wavelength for colloidal silver varies from about 390-420 nanometers depending on the size of the colloids, which gives a solution of silver colloids a characteristic yellow color. Similarly, when shell 16 includes principally gold, nanoshell 10 may have a plasmon resonance with a peak wavelength greater than about 500 nanometers to about 20 microns. In contrast, the peak wavelength for colloidal gold varies from about 500-530 nanometers depending on the size of the colloids, giving a solution of gold colloids a characteristic red color. In both cases, the nanoshell plasmon resonance is red-shifted from the corresponding colloid.

The thickness of shell 16 is defined as the difference between the outer radius and the inner radius, computed by subtracting the inner radius from the outer radius. The inner radius is half the average diameter of the inner surface and the outer radius is half the average diameter of the outer surface. In some embodiments, shell 16 has a thickness less than the bulk electron mean free path of the material primarily forming shell 16. When the thickness of shell 16 is greater than or equal to the bulk electron mean free path, that is the value of the mean free path in a bulk amount of the material forming shell 16, the shell electron mean free path is equal to the bulk electron mean free path. When the thickness of shell 16 is less than the bulk electron mean free path, the shell electron mean free path is equal to the thickness of shell 16. Thus, when the thickness of shell 16 is less than the bulk electron mean free path, size-dependent effects are present in the peak width 62.

According to some embodiments, a plurality of cores 15 and a plurality of nanoshells 10 can be substantially monodisperse. For example, in one embodiment, a plurality of cores 15 is characterized by a distribution of sizes with a standard deviation of up to about 20%, more preferably up to about 10%. Alternatively, either of a plurality of cores 15 and a plurality of nanoshells 10 may be polydisperse. Thus, in some embodiments, non-homogeneous broadening in plasmon resonance originating from a plurality of nanoshells 10 may occur in part due to polydisperse nature of nanoshells 10.

Shell 16 can be a complete shell, i.e., one which extends substantially continuously between the inner surface and the outer surface of shell 16, and completely surrounds and encapsulates core 15. When shell 16 is complete, the peak wavelength of the plasmon resonance is related to the geometry of nanoshell 10, specifically, to the ratio of the thickness of shell 16 to the size of core 15. As shell 16 increases in thickness, the peak wavelength of nanoshell 10 shifts to shorter wavelengths. Thus, the progress of a reaction forming shell 16 may be followed spectrophotometrically and terminated when a desired peak wavelength is obtained.

Alternatively, a nanoshell may include a partial shell, i.e., one which covers only a portion of a core. The portion covered preferably extends within a solid angle θ of coverage less than 360°.

A nanocup is another embodiment of nanoshell, where a shell is layered on a core, and where the shell is a partial shell extending within a solid angle θ at least 180° and less than 360°. The solid angle is more preferably between about 300° and about 350°.

A nanocap is another embodiment of nanoshell, where a shell layered on a core, where the shell is a partial shell extending within solid preferably between about 10° and about 60°.

Referring again to FIG. 1, it should be understood that core 15 may alternately be an inner composite particle that includes a solid core and at least one shell. Further, it is contemplated that a nanoshell 10 may include a core and any number of metallic shells. A metallic shell may be layered upon another metallic shell. Alternatively, a pair of metallic shells can be separated by a coating. Each shell can be a conducting or non-conducting layer. Exemplary non-conducting layers include dielectric materials and semi-conducting materials.

B. Making Metal Nanoshells

Methods for making isotropic metal nanoshells are disclosed in U.S. Pat. No. 6,342,219; U.S. Patent Application Publication Number 20020061363, filed Sep. 27, 2001; U.S. Patent Application Publication Number 20020160195 filed Nov. 5, 2001; and, U.S. application Ser. No. 10/013,259, entitled Multi-Layer Metal Nanoshells, filed Nov. 5, 2001, which are each hereby incorporated herein by reference. Various methods of making nanoshells described in these references are described below.

One method for making a nanoshell as described above, includes providing a silica core, and growing a gold shell on the silica core, using aminopropyltriethoxysilane molecules to generate linker molecules that functionalize the core. The method preferably includes first aging a solution of gold colloidal particles, from a period from about 5 to about 30 days, more preferably from about 7 to about 24 days, still more preferably from about 10 to about 20 days. The aging is preferably carried out under refrigeration, preferably at a temperature of about 40° F. (about 4° C.). Growth of the gold shell includes attaching gold colloidal particles to the linker molecules and reducing additional gold from solution onto the gold colloidal particles, preferably in solution.

Another embodiment of a process for making nanoshells, relates to growing monodisperse silica cores using the Stöber method, described in W. Stöber, et al. Journal of Colloid and Interface Science 26, pp. 62-69 (1968), hereby incorporated herein by reference. In particular, tetraethylorthosilicate (TEOS), ammonium hydroxide (NH₄OH), and water are added to a glass beaker containing ethanol, and the mixture is stirred overnight. The size of the Stöber particles is dependent on the relative concentrations of the reactants. These particles are then functionalized with 3-aminopropyltriethoxysilane (APTES). The 3-aminopropyltriethoxysilane (APTES) hydrolyzes to form a 3-aminopropylsilanetriol linker molecule. The silane group attaches to the silica surface, and the amine group is exposed.

In another exemplary process for making nanoshells, ultrasmall gold colloid (1-3 nm) is synthesized using a recipe reported by Duff, disclosed in D. G. Duff, et al., Langmuir 9, pp. 2310-2317 (1993) (Duff, et al.), hereby incorporated herein by reference. This entails, for example, a solution of 45 mL of water, 1.5 mL of 29.7 mM HAuCl₄, 300 uL of 1M NaOH and 1 mL (1.2 mL aqueous solution diluted to 100 mL with water) of tetrakishydroxymethylphosphoniumchloride (THPC). This gold is then added to the functionalized silica particles, preferably after aging as described above. The gold colloid attaches to the amine-terminated silica particles, which provide nucleation sites for the chemical deposition of a metallic shell.

It will be understood that, alternatively, any metal that can be made in colloidal form could be attached as a metal cluster. Alternative metals that may be used to form a partial shell include any suitable metals as described above, for example, silver, platinum, palladium or lead.

Further, metal nanoshells can include an intermediate layer of a functionalizing metal, which is preferably tin or titanium. Tin functionalization is described in U.S. Patent Application Publication Number 20020061363, filed Sep. 27, 2002. As disclosed therein, functionalization with gold colloid attached to a linker molecule attached to a substrate, as described above, may be replaced by tin functionalization. In this way, nanoshells each having a layer of a shell metal, may be made by mixing tin ions and substrate particles in solution to form functionalized particles, followed by reduction of the shell metal onto the functionalized particles.

To perform this process, after separation from a reactant solution, such as by centrifugation, Stöber particles are redispersed in a first solvent and submerged in a solution of SnCl₂ in a second solvent. The solvents may be water, or more preferably, a methanol/water mixture, preferably 50% by volume methanol. A solution of tin chloride in a methanol/water solvent preferably includes a surfactant, such as CF₃COOH. A method of tin functionalization using a methanol/water solvent is described, for example in Yoshio Kobayashi, et al. Chemical Materials 13, pp. 1630-1633 (2001), hereby incorporated herein by reference. By adding tin (II) chloride SnCl₂ and Stöber nanoparticles in a solvent, it is believed that tin atoms are deposited chemically onto the surface of the Stöber nanoparticles. Small tin precursor particles (<2 nm) form on the surface of the silica nanoparticle upon addition of more SnCl₂ to the solution.

After a period of time, preferably at least 45 minutes, the tin-functionalized silica particles are separated from solution and redispersed in water. The separation from solution is achieved on the lab bench scale by centrifugation. Centrifugation has the advantage of removing any excess tin and preparing the tin-coated nanoparticles for further metal reduction. When the functionalized particles are redisbursed in water the pH tends to drop to about 3. The pH is preferably raised to at least 9 for subsequent reduction of silver, which achieves reaction conditions favorable for reduction of a shell metal.

Reduction of shell metal includes mixing a functionalized dielectric substrate, a plurality of metal ions and a reducing agent in solution. Formaldehyde is a preferred reducing agent. The metal may be any shell metal as disclosed above.

When the metal is selected from among silver, copper, and nickel, the method preferably further includes raising the pH of the solution to more effectively coat the substrate with the metal. In particular, in one embodiment, gold-functionalized silica particles are mixed with 0.15 mM solution of fresh silver nitrate and stirred vigorously. A small amount (typically 25-50 microliters) of 37% formaldehyde is added to begin the reduction of the silver ions onto the gold particles on the surface of the silica. This step is followed by the addition of doubly distilled ammonium hydroxide (typically 50 microliters). The “amounts” or “relative amounts” of gold-functionalized silica and silver nitrate dictate the core to shell ratio and hence the absorbance. Before further use, the nanoshell solution is preferably centrifuged to separate the nanoshells from solution and remove byproducts and any solid silver colloid that formed. The nanoshells are preferably resuspended in a solvent, e.g., water or ethanol. Cycles of centrifugation and resuspension may be repeated until the resuspended solution is sufficiently pure.

C. Targeting Molecules

As noted above, suitable targeting molecules include antibodies, antibody fragments, antibodies, antibody fragments, receptor binding proteins or other proteins or molecules including growth factors, including those which target receptors and proteins expressed on the surface of the endothelial cells of the neovasculature. Such targeting molecules may target the VEGF receptor or one or more of the variants thereof or other cell-surface receptors. Retinal blood vessels subjected to ischemic stress have many more receptors for VEGF than vessels elsewhere. This characteristic allows targeting molecules directed to the VEGF receptor, and the nanoshells conjugated thereto, to accumulate in the retina at a higher concentration than in other tissues.

Anti-VEGF receptor monoclonal antibodies and methods of making them are disclosed, for example, in U.S. Pat. Nos. 6,344,339 and 6,448,077, the latter of which discloses that hybridoma cell lines producing such monoclonal antibodies were deposited at the ATCC, Manassas, Va., as ATCC Accession Nos. HB 11534: HB12152; and HB-12153. Such antibodies can be conjugated to the nanoshells of the invention, using the methods set forth below, for use in targeting the nanoshells to the eye. An alternative method of targeting the nanoshells is by conjugating them with VEGF itself. This molecule will target its receptor and bring the nanoshell into proximity with the neovasculature in the eye.

Rather than targeting VEGF receptor, the targeting molecules could also be against other molecules associated with angiogenesis. The endothelial adhesion receptor of integrin alpha v3 is known to provide a vasculature-specific target for anti-angiogenic treatment strategies. See Brooks, P. C., Clark, R. A. & Cheresh, D. A. (1994) “Requirement of vascular integrin alpha v beta 3 for angiogenesis”, Science 264, 569-571; Friedlander, M., et. al., (1995); “Definition of two angiogenic pathways by distinct alpha v integrins”, Science 270, 1500-1502. The requirement for vascular integrin aVss3 in angiogenesis was demonstrated by several in vivo models where the generation of new blood vessels by transplanted human tumors was entirely inhibited either by systemic administration of peptide antagonists of integrin aVss3 or anti-aVss3 antibody LM609. Murine hybridoma LM609 was deposited with the ATCC, Manassas, Va., under Accession No. HB 9537. (Brooks, P. C., et. al., (1994) Science supra; Brooks, P. C., et. al., (1994) “Integrin alpha v beta 3 antagonists promote tumor regression by inducing apoptosis of angiogenic blood vessels,” Cell 79,1157-1164). Accordingly, aVss3 would be a suitable target for a targeting molecule, for example, a monoclonal antibody, including LM609 or others, or proteins binding to aVss3.

By way of background, it is noted that monoclonal antibodies are antibodies (or immunoglobulins) derived from a single clone of B-lymphocytes. These B cells are immortalized to provide a cell line able to indefinitely produce antibodies which are all specific to a particular target antigen.

In a conventional process for making monoclonal antibodies, a mouse is immunized with an antigen of interest (including, for example, VEGF receptor or aVss3), and its immune system is boosted with adjuvants so that it generates an enhanced response against the immunogen. The mouse B lymphocytes are extracted from the mouse spleens (which contain high numbers of B-lymphocytes), and then fused with an immortal myeloma cell line. Some of the hybridomas resulting from the fusion produce monoclonal antibodies to the antigen initially used to immunize the mouse. The hybridomas secreting antibodies with the desired characteristics (e.g., anti-VEGF receptor or anti-aVss3) are selected.

Mouse-derived portions of a monoclonal antibody can cause immune reactions against the antibody upon human therapeutic use (called a human anti-mouse or “HAMA” response), especially when there is repeated dosing. This can lead to adverse patient consequences, in the worst case scenario, or, otherwise, a need for higher dosages as the antibody is targeted by the patient's immune system and removed.

Several genetic engineering technologies have been developed to reduce the amount of mouse protein in an antibody and to make as much as possible human-derived. See, e.g., L. Riechmann et al., Nature (1988); 332: 323-327; U.S. Pat. No. 5,225,539; U.S. Pat. No. 5,530,101. DEIMMUNISED™ antibodies are antibodies in which the T and B cell epitopes have been eliminated using genetic engineering, as described in International Patent Application WO9852976. They are designed to have reduced immunogenicity when applied in vivo. Antibody fragments are smaller and therefore have less mouse protein than whole antibodies, and therefore, are likely to be less immunogenic. Antibody fragments include Fab, F(ab′)₂, and Fd fragments. These fragments can be isolated from antibody phage libraries generated using the techniques described in McCafferty et al., Nature 348:552-554 (1990), Clackson et al., Nature 352:624-628 (1991) and Marks et al., J. Mol. Biol. 222:581-597 (1991). Subsequent publications describe the production of high affinity (nM range) human antibodies by chain shuffling (Marks et al., Bio/Technology 10:779-783 (1992)), as well as combinatorial infection and in vivo recombination as a strategy for constructing very large phage libraries (Waterhouse et al., Nuc. Acids. Res. 21:2265-2266 (1993)). Single chain Fv molecules are another binding molecule which can be made using phage display techniques. See U.S. Pat. No. 5,565,332 and European Patent No. 0 589 877 B1. Again, they are smaller and have less mouse proteins than whole mouse antibodies.

Some companies (notably, Abgenix, Inc., Fremont, Calif., and Medarex, Inc., Annandale, N.J.) have genetically re-engineered mice themselves, so that the mice produce substantially human antibodies. Antibody-producing cells from such mice are then immortalized to make monoclonal antibodies.

All such monoclonal antibodies, including mouse, but preferably, chimeric, humanized, DEIMMUNISED™ and human antibodies, as well as antibody fragments and single chain Fv molecules, are suitable for use as targeting agents in the present invention. It is also possible to screen for other types of proteins and molecules which bind to the antigens of interest, i.e., VEGF receptor and aVss3, using well-known techniques, and use such proteins or molecules as the targeting agent.

D. Conjugation of Metal Nanoshells and Targeting Molecules

Nanoshells and targeting molecules may be conjugated using several methods, including covalent or ionic bonding. Covalent bonding of nanoparticles to proteins is described in U.S. Patent Application Publication No. 20020015679, filed May 31, 2001, incorporated by reference. This application describes conjugation of a thiol stabilizer with a nanoparticle, which is in turn conjugated with a protein or antibody. Exemplary thiol stabilizers include thioglycerol (OH), mercaptosuccinic acid (—COOH), thioglycolic acid (—COOH), and 1-amino-2-methyl-2-propanethiol (—NH₂) (the terminal functional group is indicated in parenthesis). In a reaction in which the terminal functional group is activated, a protein, antibody or antibody fragment can be bound to the thiol stabilizer through the active group, and thus to the nanoshell.

Another method for binding proteins or antibodies to nanoshells is analogous to that disclosed in U.S. Pat. No. 4,472,509, incorporated by reference, wherein diethylenetriaminepentaacetic acid (DTPA) chelating agents are used to bind radiometals to monoclonal antibodies. An antibody is reacted with a quantity of a selected bifunctional chelating agent having metal binding functionalities to produce a chelator/antibody conjugate. In conjugating the antibodies with the chelators an excess of chelating agent is reacted with the antibodies, the specific ratio being dependent upon the nature of the reagents and the desired number of chelating agents per antibody. The purified chelator/antibody conjugate may then be chelated with the metal nanoshell, preferably in an aqueous solution with pH generally ranging from about 3.2 to about 9 so as not to impair the biological activity or specificity of the antibodies.

The metal nanoshell may also be conjugated with polyethylene glycol to improve the stability of the metal nanoshell in biological fluids. Alternatively, to improve stability of the nanoshell conjugate, the targeting molecule can be conjugated to one end of the polyethylene glycol.

E. Application of the Invention

Conjugated nanoshells can be delivered to the target area, e.g., the eye, by local injection or by systemic delivery. Alternatively, the target area may be any area characterized by excessive or inappropriate angiogenesis. Once the conjugated nanoshells are at the target, an infrared laser is used to irradiate the nanoshells, preferably at a wavelength which is at or close to the plasmon resonance of the nanoshells. The heat generated on irradiation ablates or disrupts the blood vessels, arresting angiogenesis or ameliorating the effects of the neovasculature on the vision.

The dosage levels of the nanoshell conjugates for use in treatment can be arrived at by several well-known methods. One method involves extrapolation from animal disease models. For example, based on the relative sizes, one can extrapolate the human does from experiments demonstrating the amount needed to effectively treat a small mammal, such as a mouse. The dosage is, as with other treatments, then further refined in the course of human clinical trials.

The dosage from patient to patient could also vary based on a number of factors, particularly including the number of target molecules at the disease site, and the amount of neovasculature. Additionally, it may be appropriate to perform a series of treatments over time, each with a smaller dosage than would be given for a single dose treatment.

Compositions suitable for administration by injection include the conjugated nanoshells dispersed in a pharmaceutically acceptable carrier, which can include any and/or all solvents, dispersion media, coatings, antibacterial and/or antifungal agents, isotonic and/or absorption delaying agents, except to the extent such agents are incompatible with other composition ingredients.

Administration can be by parenteral administration, e.g., it can be formulated for injection via the intravenous, intramuscular, sub-cutaneous, intralesional, and/or even intraperitoneal routes. Compositions suitable for injectable use include sterile aqueous dispersions; formulations including sesame oil, peanut oil and/or aqueous propylene glycol; and/or sterile powders for the extemporaneous preparation of sterile injectable solutions and/or dispersions. The composition must be sterile and fluid so it can be injected. It can also include antibacterial and antifungal agents, parabens, chlorobutanol, phenol, sorbic acid and thimerosal. In many cases, it will be preferable to include isotonic agents, for example, sugars and/or sodium chloride.

For parenteral administration in an aqueous dispersion or solution, for example, it should be suitably buffered if necessary and/or the liquid diluent first rendered isotonic with sufficient saline and/or glucose. These particular aqueous dispersions and solutions are especially suitable for intravenous, intra-arterial, intramuscular, subcutaneous and/or intraperitoneal administration.

Because of the proximity to the eyes, one may also administer using ophthalmic solutions, nasal solutions and/or sprays, aerosols and/or inhalants. The nanoshells of the present invention can be administered by many routes and are amenable to most common pharmaceutical preparations.

It should be understood that the terms and expressions used herein are exemplary only and not limiting, and that the scope of the invention is defined only in the claims which follow, and includes all equivalents of such claims. The term “Monoclonal Antibody” as used in the claims refers to all monoclonal antibodies and derivatives and fragments thereof having binding activity, including but not limited to mouse, humanized, human, and DEIMMUNISED™ antibodies, and fragments including Fab, F(ab′)₂, and Fd fragments, and single chain Fv binding molecules.

What is claimed is:

1. A method of reducing or inhibiting excessive or inappropriate neovasculature comprising:
admininstering a conjugate or chelate comprising a nanoparticle and a targeting agent, wherein said agent targets the neovasculature or cells or tissues proximal to the neovasculature; and
irradiating the area of the neovasculature to heat the nanoparticles sufficiently to damage the neovasculature.

2. The method of claim 1 wherein the irradiation is with a laser which emits a wavelength at or near the plasmon resonance of the nanoparticle.

3. The method of claim 2 wherein the wavelength emitted is between 700 and 1300 nm.

4. The method of claim 3 wherein the wavelength is between 750 and 1100 nm.

5. The method of claim 1 wherein the targeting agent is VEGF, a Monoclonal Antibody targeting the VEGF receptor, or a Monoclonal Antibody targeting aVss3.

6. The method of claim 1 wherein the targeting agent is conjugated to the nanoparticle through a conjugating agent.

7. The method of claim 6 wherein the conjugating agent is one or more of polyethylene glycol, thioglycerol, mercaptosuccinic acid, thioglycolic acid or 1-amino-2-methyl-2-propanethiol.

8. The method of claim 1 wherein the targeting agent is chelated with the nanoparaticle with the chelating agent diethylenetriaminepentaacetic acid.

9. The method of claim 1 wherein the nanoparticle is a nanoshell, a metal colloid, a fullerene, a nanotube, or a derivatized nanotube or a derivatized fullerene.

10. The method of claim 9 wherein the nanoshell has a core material that is dielectric or semiconducting and a shell material that is conducting.

11. The method of claim 9 wherein the nanoshells have a silica core and the shell is metal.

12. The method of claim 11 wherein the metal is gold.

13. A method of treating reducing or inhibiting excessive or inappropriate neovasculature in the eye associated with diabetic retinopathy or macular degeneration comprising:
administering a nanoparticle which is targeted to a region of neovasculature in the eye;
waiting for a sufficient time for the nanoparticle to arrive at the target area; and
irradiating the region so as to heat the region and damage the neovasculature.

14. The method of claim 13 wherein the administration is by intravenous or intra-arterial injection.

15. The method of claim 13 further including repeating the method steps if neovasculature appears in the same subject following the initial treatment.

16. The method of claim 13 wherein the irradiation is with a laser which emits a wavelength at or near the plasmon resonance of the nanoparticle.

17. The method of claim 16 wherein the wavelength emitted is 700 and 1300 nm.

18. The method of claim 17 wherein the wavelength is between 750 and 1100 nm.

19. The method of claim 13 wherein the nanoparticle is targeted by linking it to a targeting agent, which is VEGF, a Monoclonal Antibody targeting the VEGF receptor, or a Monoclonal Antibody targeting aVss3.

20. The method of claim 13 wherein the targeting agent is conjugated to the metal nanoshell through a conjugating agent.

21. The method of claim 20 wherein the conjugating agent is one or more of polyethylene glycol, thioglycerol, mercaptosuccinic acid, thioglycolic acid or 1-amino-2-methyl-2-propanethiol.

22. The method of claim 20 wherein the targeting agent is chelated with the nanoparticle with diethylenetriaminepentaacetic acid.

23. The method of claim 13 wherein the nanoparticle is a metal nanoshell which includes a core material that is dielectric or semiconducting and a shell material that is conducting.

24. The method of claim 23 wherein the nanoshell has a silica core and the shell is metal.

25. The method of claim 24 wherein the metal is gold.
